SEQUENCE LISTING (9 sequences)

SEQ ID NO: 1 - DNA, 4080 bp, Corynebacterium glutamicum
  gene (288)..(1169): sdhC
gtgcccggcgtggtcgggccacatccgccccgggaactttttaggcacctacggtgcaactgttgggataattgtgtcacctgcgcaaagttgctccctggatcggaaggttgggctgtctaaactttttggttgataccaaacggggttagaaactgttcggatcggtatcctgtgaggaagctcaccttggttttagaatgttgaaaaggcctcacgtttccgcaggtagagcacactcaattaaatgagcgtcaaacgacaataaagtaaggctatcctaataagtggggttttatgtctctaaacagccagttgggggtcatgggggagcgccccgtgactggttaatgccccgatctgggacgtacagtaacaacgacactggaggtgccatgactgttagaaatcccgaccgtgaggcaatccgtcacggaaaaattacgacggaggcgctgcgtgagcgtcccgcatacccgacctgggcaatgaagctgaccatggccatcactggcctaatgtttggtggcttcgttcttgttcacatgatcggaaacctgaaaatcttcatgccggactacgcagccgattctgcgcatccgggtgaagcacaagtagatgtctacggcgagttcctgcgtgagatcggatccccgatcctcccacacggctcagtcctctggatcctacgtattatcctgctggtcgcattggttctgcacatctactgtgcattcgcattgaccggccgttctcaccagtcccgcggaaagttccgccgtaccaacctcgttggcggcttcaactccttcgcgacccgctccatgctggtgaccggaatcgttctccttgcgttcattatcttccacatcctcgacctgaccatgggtgttgctccagcagccccaacctcattcgagcacggcgaagtatacgcaaacatggtggcttcctttagccgctggcctgtagcaatttggtacatcattgccaacctggtcctgttcgtccacctgtcacacggcatctggcttgcagtctctgacctgggaatcaccggacgccgctggagggcaatcctcctcgcagttgcgtacatcgttcctgcactggtcctgatcggcaacatcaccattccgttcgccatcgctgttggctggattgcgtaaaggttaggaagaatttatgagcactcactctgaaaccacccgcccagagttcatccacccagtctcagtcctcccagaggtctcagctggtacggtccttgacgctgcagagccagcaggcgttcccaccaaagatatgtgggaataccaaaaagaccacatgaacctggtctccccactgaaccgacgcaagttccgtgtcctcgtcgttggcaccggcctgtccggtggtgctgcagcagcagccctcggcgaactcggatacgacgtcaaggcgttcacctaccacgacgcacctcgccgtgcgcactccattgctgcacagggtggcgttaactccgcccgcggcaagaaggtagacaacgacggcgcataccgccacgtcaaggacaccgtcaagggcggcgactaccgtggtcgcgagtccgactgctggcgtctcgccgtcgagtccgtccgcgtcatcgaccacatgaacgccatcggtgcaccattcgcccgcgaatacggtggcgccttggcaacccgttccttcggtggtgtgcaggtctcccgtacctactacacccgtggacaaaccggacagcagctgcagctctccaccgcatccgcactacagcgccagatccacctcggctccgtagaaatcttcacccataacgaaatggttgacgtcattgtcaccgaacgtaacggtgaaaagcgctgcgaaggcctgatcatgcgcaacctgatcaccggcgagctcaccgcacacaccggccatgccgttatcctggcaaccggtggctacggcaacgtgtaccacatgtccaccctggccaagaactccaacgcctcggccatcatgcgtgcatacgaagccggcgcatacttcgcgtccccatcgttcatccagttccacccaaccggcctgcctgtgaactccacctggcagtccaagaccattctgatgtccgagtcgctgcgtaacgacggccgcatctggtcccctaaggaaccgaacgataaccgcgatccaaacaccatccctgaggatgagcgcgactacttcctggagcgccgctacccagcattcggtaacctcgtcccacgtgacgttgcttcccgtgcgatctcccagcagatcaatgctggtctcggtgttggacctctgaacaacgctgcatacctggacttccgcgacgccaccgagcgcctcggacaggacaccatccgcgagcgttactccaacctcttcaccatgtacgaagaggcaattggcgaggacccatactccagcccaatgcgtattgcaccgacctgccacttcaccatgggtggcctctggactgacttcaacgaaatgacgtcactcccaggtctgttctgcgcaggcgaagcatcctggacctaccacggtgcaaaccgtctgggcgcaaactccctgctctccgcttccgtcgatggctggttcaccctgccattcaccatccctaactacctcggcccattgcttggctccgagcgtctgtcagaggatgcaccagaagcacaggcagcgattgcgcgtgcacaggctcgcattgaccgcctcatgggcaaccgcccagagtgggtcggtgacaacgttcacggacctgagtactaccaccgccagcttggcgatatcctgtacttctcctgtggcgtttcccgaaacgtagaagacctccaggatggcatcaacaagatccgtgccctccgcgatgacttctggaagaacatgcgcatcaccggcagcaccgatgagatgaaccaggttctcgaatacgcagcacgcgtagccgactacatcgacctcggcgaactcatgtgtgtcgacgccctcgaccgcgacgagtcctgtggcgctcacttccgcgacgaccacctctccgaagatggcgaagcagaacgtgacgacgaaaactggtgcttcgtctccgcatgggaaccaggcgagaacggaaccttcgtccgccacgcagaaccactgttcttcgaatccgtcccactgcagacaaggaactacaagtaatgaaacttacacttgagatctggcgtcaagcaggcccaactgcggaaggcaagttcgaaaccgtccaggttgacgacgccgtcgcgcagatgtccatcctggagctgcttgaccacgtaaacaacaagttcatcgaagaaggcaaagaaccattcgcgttcgcctctgactgccgcgaaggcatttgtggtacctgtggtctcctcgtgaacggtcgccctcacggcgccgaccagaacaagcctgcctgtgcgcagcgcctggtcagctacaaggaaggcgacaccctcaagatcgaaccactgcgttccgccgcatacccagtgatcaaggacatggtcgtcgaccgctccgcactggaccgtgtcatggaacagggtggctacgtgaccatcaacgcaggtaccgcacctgacgctgataccctccacgtcaaccacgaaaccgcagaactcgcacttgaccacgcagcctgcatcggctgtggcgcatgtgttgctgcctgccctaacggcgcagcacacctgttcaccggcgcaaagcttgttcacctctccctcctcccactgggtaaggaagagcgcggactgcgtgcacgtaagatggttgatgaaatggaaaccaacttcggacactgctccctctacggcgagtgcgcagatgtctgccccgcaggcatcccactgaccgctgtggcagctgtcaccaaggaacgtgcgcgtgcagctttccgaggcaaagacgactagtctttaatccaagtaagtaccggttcagacagttaaaccagaaagacgagtgaacaccatgtcctccgcgaaaaagaaacccgcaccggagcgtatgcactacatcaagggctatgtacctgtg

SEQ ID NO: 2 - DNA, 882 bp, Corynebacterium glutamicum
  CDS (1)..(879): sdhC Gene
gtggggttttatgtctctaaacagccagttgggggtcatgggggagcgccccgtgactggttaatgccccgatctgggacgtacagtaacaacgacactggaggtgccatgactgttagaaatcccgaccgtgaggcaatccgtcacggaaaaattacgacggaggcgctgcgtgagcgtcccgcatacccgacctgggcaatgaagctgaccatggccatcactggcctaatgtttggtggcttcgttcttgttcacatgatcggaaacctgaaaatcttcatgccggactacgcagccgattctgcgcatccgggtgaagcacaagtagatgtctacggcgagttcctgcgtgagatcggatccccgatcctcccacacggctcagtcctctggatcctacgtattatcctgctggtcgcattggttctgcacatctactgtgcattcgcattgaccggccgttctcaccagtcccgcggaaagttccgccgtaccaacctcgttggcggcttcaactccttcgcgacccgctccatgctggtgaccggaatcgttctccttgcgttcattatcttccacatcctcgacctgaccatgggtgttgctccagcagccccaacctcattcgagcacggcgaagtatacgcaaacatggtggcttcctttagccgctggcctgtagcaatttggtacatcattgccaacctggtcctgttcgtccacctgtcacacggcatctggcttgcagtctctgacctgggaatcaccggacgccgctggagggcaatcctcctcgcagttgcgtacatcgttcctgcactggtcctgatcggcaacatcaccattccgttcgccatcgctgttggctggattgcgtaa

SEQ ID NO: 3 - protein, 293 aa, Corynebacterium glutamicum
VGFYVSKQPVGGHGGAPRDWLMPRSGTYSNNDTGGAMTVRNPDREAIRHGKITTEALRERPAYPTWAMKLTMAITGLMFGGFVLVHMIGNLKIFMPDYAADSAHPGEAQVDVYGEFLREIGSPILPHGSVLWILRIILLVALVLHIYCAFALTGRSHQSRGKFRRTNLVGGFNSFATRSMLVTGIVLLAFIIFHILDLTMGVAPAAPTSFEHGEVYANMVASFSRWPVAIWYIIANLVLFVHLSHGIWLAVSDLGITGRRWRAILLAVAYIVPALVLIGNITIPFAIAVGWIA

SEQ ID NO: 4 - DNA, 1878 bp, Corynebacterium glutamicum
  CDS (1)..(1875): sdhA gene
atgaacctggtctccccactgaaccgacgcaagttccgtgtcctcgtcgttggcaccggcctgtccggtggtgctgcagcagcagccctcggcgaactcggatacgacgtcaaggcgttcacctaccacgacgcacctcgccgtgcgcactccattgctgcacagggtggcgttaactccgcccgcggcaagaaggtagacaacgacggcgcataccgccacgtcaaggacaccgtcaagggcggcgactaccgtggtcgcgagtccgactgctggcgtctcgccgtcgagtccgtccgcgtcatcgaccacatgaacgccatcggtgcaccattcgcccgcgaatacggtggcgccttggcaacccgttccttcggtggtgtgcaggtctcccgtacctactacacccgtggacaaaccggacagcagctgcagctctccaccgcatccgcactacagcgccagatccacctcggctccgtagaaatcttcacccataacgaaatggttgacgtcattgtcaccgaacgtaacggtgaaaagcgctgcgaaggcctgatcatgcgcaacctgatcaccggcgagctcaccgcacacaccggccatgccgttatcctggcaaccggtggctacggcaacgtgtaccacatgtccaccctggccaagaactccaacgcctcggccatcatgcgtgcatacgaagccggcgcatacttcgcgtccccatcgttcatccagttccacccaaccggcctgcctgtgaactccacctggcagtccaagaccattctgatgtccgagtcgctgcgtaacgacggccgcatctggtcccctaaggaaccgaacgataaccgcgatccaaacaccatccctgaggatgagcgcgactacttcctggagcgccgctacccagcattcggtaacctcgtcccacgtgacgttgcttcccgtgcgatctcccagcagatcaatgctggtctcggtgttggacctctgaacaacgctgcatacctggacttccgcgacgccaccgagcgcctcggacaggacaccatccgcgagcgttactccaacctcttcaccatgtacgaagaggcaattggcgaggacccatactccagcccaatgcgtattgcaccgacctgccacttcaccatgggtggcctctggactgacttcaacgaaatgacgtcactcccaggtctgttctgcgcaggcgaagcatcctggacctaccacggtgcaaaccgtctgggcgcaaactccctgctctccgcttccgtcgatggctggttcaccctgccattcaccatccctaactacctcggcccattgcttggctccgagcgtctgtcagaggatgcaccagaagcacaggcagcgattgcgcgtgcacaggctcgcattgaccgcctcatgggcaaccgcccagagtgggtcggtgacaacgttcacggacctgagtactaccaccgccagcttggcgatatcctgtacttctcctgtggcgtttcccgaaacgtagaagacctccaggatggcatcaacaagatccgtgccctccgcgatgacttctggaagaacatgcgcatcaccggcagcaccgatgagatgaaccaggttctcgaatacgcagcacgcgtagccgactacatcgacctcggcgaactcatgtgtgtcgacgccctcgaccgcgacgagtcctgtggcgctcacttccgcgacgaccacctctccgaagatggcgaagcagaacgtgacgacgaaaactggtgcttcgtctccgcatgggaaccaggcgagaacggaaccttcgtccgccacgcagaaccactgttcttcgaatccgtcccactgcagacaaggaactacaagtaa

SEQ ID NO: 5 - protein, 625 aa, Corynebacterium glutamicum
MNLVSPLNRRKFRVLVVGTGLSGGAAAAALGELGYDVKAFTYHDAPRRAHSIAAQGGVNSARGKKVDNDGAYRHVKDTVKGGDYRGRESDCWRLAVESVRVIDHMNAIGAPFAREYGGALATRSFGGVQVSRTYYTRGQTGQQLQLSTASALQRQIHLGSVEIFTHNEMVDVIVTERNGEKRCEGLIMRNLITGELTAHTGHAVILATGGYGNVYHMSTLAKNSNASAIMRAYEAGAYFASPSFIQFHPTGLPVNSTWQSKTILMSESLRNDGRIWSPKEPNDNRDPNTIPEDERDYFLERRYPAFGNLVPRDVASRAISQQINAGLGVGPLNNAAYLDFRDATERLGQDTIRERYSNLFTMYEEAIGEDPYSSPMRIAPTCHFTMGGLWTDFNEMTSLPGLFCAGEASWTYHGANRLGANSLLSASVDGWFTLPFTIPNYLGPLLGSERLSEDAPEAQAAIARAQARIDRLMGNRPEWVGDNVHGPEYYHRQLGDILYFSCGVSRNVEDLQDGINKIRALRDDFWKNMRITGSTDEMNQVLEYAARVADYIDLGELMCVDALDRDESCGAHFRDDHLSEDGEAERDDENWCFVSAWEPGENGTFVRHAEPLFFESVPLQTRNYK

SEQ ID NO: 6 - DNA, 855 bp, Corynebacterium glutamicum
  CDS (1)..(852): sdhB Gene
gtgcttcgtctccgcatgggaaccaggcgagaacggaaccttcgtccgccacgcagaaccactgttcttcgaatccgtcccactgcagacaaggaactacaagtaatgaaacttacacttgagatctggcgtcaagcaggcccaactgcggaaggcaagttcgaaaccgtccaggttgacgacgccgtcgcgcagatgtccatcctggagctgcttgaccacgtaaacaacaagttcatcgaagaaggcaaagaaccattcgcgttcgcctctgactgccgcgaaggcatttgtggtacctgtggtctcctcgtgaacggtcgccctcacggcgccgaccagaacaagcctgcctgtgcgcagcgcctggtcagctacaaggaaggcgacaccctcaagatcgaaccactgcgttccgccgcatacccagtgatcaaggacatggtcgtcgaccgctccgcactggaccgtgtcatggaacagggtggctacgtgaccatcaacgcaggtaccgcacctgacgctgataccctccacgtcaaccacgaaaccgcagaactcgcacttgaccacgcagcctgcatcggctgtggcgcatgtgttgctgcctgccctaacggcgcagcacacctgttcaccggcgcaaagcttgttcacctctccctcctcccactgggtaaggaagagcgcggactgcgtgcacgtaagatggttgatgaaatggaaaccaacttcggacactgctccctctacggcgagtgcgcagatgtctgccccgcaggcatcccactgaccgctgtggcagctgtcaccaaggaacgtgcgcgtgcagctttccgaggcaaagacgactag

SEQ ID NO: 7 - protein, 284 aa, Corynebacterium glutamicum
VLRLRMGTRRERNLRPPRRTTVLRIRPTADKELQVMKLTLEIWRQAGPTAEGKFETVQVDDAVAQMSILELLDHVNNKFIEEGKEPFAFASDCREGICGTCGLLVNGRPHGADQNKPACAQRLVSYKEGDTLKIEPLRSAAYPVIKDMVVDRSALDRVMEQGGYVTINAGTAPDADTLHVNHETAELALDHAACIGCGACVAACPNGAAHLFTGAKLVHLSLLPLGKEERGLRARKMVDEMETNFGHCSLYGECADVCPAGIPLTAVAAVTKERARAAFRGKDD

SEQ ID NO: 8 - DNA, 20 bp, artificial sequence
  Primer for sdhA gene amplification via PCR
cgtcattgtcaccgaacgta

SEQ ID NO: 9 - DNA, 20 bp, Artificial Sequence
  Primer for sdhA emplification via PCR
tcgttgaagtcagtccagag

(19) United States
(12) Patent Application Publication
(10) Pub. No.: US 2003/0100079 A1
(43) Pub. Date: May 29, 2003

(54) Novel nucleotide sequences coding for the genes sdhA, sdhB and sdhC

(76) Inventors: Bettina Mockel, Dusseldorf (DE); Walter Pfefferle, Halle (DE); Achim Marx, Bielefeld (DE)

Correspondence Address: Smith, Gambrell & Russell, LLP, Beveridge, DeGrandi, Weilacher & Young, Intellectual Property Group, 1850 M Street, N.W., Suite 800, Washington, DC, 20036, US

(21) Appl. No.: 09/732,923

(22) Filed: Dec. 11, 2000

(30) Foreign Application Priority Data
Dec. 10, 1999 (DE) 199 59 650.6

(51) Int. Cl. C12P 13/04; C12N 1/21; C07H 21/04

(52) U.S. Cl. 435/106; 435/252.3; 536/23.2

(57) ABSTRACT

This invention relates to isolated polynucleotides containing a polynucleotide sequence related to selected SEQ ID nos and to processes for the fermentative production of L-amino acids with attenuation of the sdhA, sdhB or sdhC gene which code for subunit A, B or C of the enzyme succinate dehydrogenase.

The present invention provides nucleotide sequences from coryneform bacteria which code for the genes sdhC, sdhA and sdhB and a process for the fermentative production of L-amino acids, in particular L-lysine, by attenuation of the sdhC and/or sdhA and/or sdhB gene. All references cited herein are expressly incorporated by reference throughout the disclosure. Incorporation by reference is also designated by the term “I.B.R.” following any citation.

### PRIOR ART

L-amino acids, in particular lysine, are used in human medicine and in the pharmaceuticals industry, in the food industry and particularly in animal nutrition.

It is known that L-amino acids are produced by fermentation of strains of coryneform bacteria, in particular Corynebacterium glutamicum. Due to their great significance, efforts are constantly being made to improve the production process. Improvements to the process may relate to measures concerning fermentation technology, for example stirring and oxygen supply, or to the composition of the nutrient media, such as for example sugar concentration during fermentation, or to working up of the product by, for example, ion exchange chromatography, or to the intrinsic performance characteristics of the microorganism itself.

The performance characteristics of these microorganisms are improved using methods of mutagenesis, selection and mutant selection. In this manner, strains are obtained which are resistant to antimetabolites or are auxotrophic for regulatorily significant metabolites and which produce L-amino acids.

For some years, methods of recombinant DNA technology have also been used to improve strains of Corynebacterium which produce L-amino acids.

### OBJECT OF THE INVENTION

An object of the invention is to provide novel measures for the improved fermentative production of amino acids, in particular L-lysine. L-amino acids, in particular lysine, are used in human medicine and in the pharmaceuticals industry, in the food industry and very particularly in animal nutrition. There is accordingly general interest in providing novel improved processes for the production of L-amino acids, in particular L-lysine.

### SUMMARY OF THE INVENTION

The present invention provides an isolated polynucleotide containing a polynucleotide sequence selected from the group

a) polynucleotide which is at least 70% identical to a polynucleotide which codes for a polypeptide containing amino acid sequence from SEQ ID no. 3,

b) polynucleotide which is at least 70% identical to a polynucleotide which codes for a polypeptide containing amino acid sequence of SEQ ID no. 5,

c) polynucleotide which is at least 70% identical to a polynucleotide which codes for a polypeptide containing amino acid sequence of SEQ ID no.7,

d) polynucleotide which codes for a polypeptide which contains an amino acid sequence which is at least 70% identical to the amino acid sequence of SEQ ID no. 3,

e) polynucleotide which codes for a polypeptide which contains an amino acid sequence which is at least 70% identical to amino acid sequence of SEQ ID no. 5,

f) polynucleotide which codes for a polypeptide which contains an amino acid sequence which is at least 70% identical to the amino acid sequence of SEQ ID no. 7,

g) polynucleotide which is complementary to the polynucleotides of a), b), c), d), e) or f) and

h) polynucleotide containing at least 15 successive nucleotides of the polynucleotide sequence of a), b), c), d), e) or f).

The present invention also provides a polynucleotide which is a preferably recombinant DNA replicable in coryneform bacteria, in particular codes for a polypeptide which contains the amino acid sequence shown in SEQ ID no. 2.

The present invention also provides a polynucleotide which is an RNA.

The present invention also provides a polynucleotide as described above, wherein it preferably comprises a replicable DNA containing:

(i) the nucleotide sequence shown in SEQ ID no. 1, or

(ii) at least one sequence which matches the sequence (i) within the degeneration range of the genetic code, or

(iii) at least one sequence which hybridizes with the complementary sequence to sequence (i) or (ii) and optionally

(iv) functionally neutral sense mutations in (i).

The present invention also provides a vector containing one of the stated polynucleotides and coryneform bacteria acting as the host cell, which contains the vector.

The present invention also provides polynucleotides which substantially consist of a polynucleotide sequence, which are obtainable by screening by means of hybridisation of a suitable gene library, which contains the complete gene having the polynucleotide sequence according to SEQ ID no. 1, with a probe which contains the sequence of the stated polynucleotide according to SEQ ID no. 1, or a fragment thereof, and isolation of the stated DNA sequence.

Polynucleotide sequences according to the invention are suitable as hybridization probes for RNA, cDNA and DNA in order to isolate full length cDNA which code for succinate dehydrogenase or the subunits A, B or C thereof and to isolate such cDNA or genes, the sequence of which exhibits a high level of similarity with that of genes for succinate dehydrogenase or the subunits A, B or C thereof. Polynucleotide sequences according to the invention are furthermore suitable as primers for the production of DNA of genes, which code for succinate dehydrogenase by the polymerase chain reaction (PCR).

Such oligonucleotides acting as probes or primers contain at least 30, preferably at least 20, very particularly preferably at least 15 successive nucleotides. Oligonucleotides having a length of at least 40 or 50 nucleotides are also suitable.

### BRIEF DESCRIPTION OF THE FIGURES

FIG. 1: Map of the plasmid pCRBluntsdhAint

### DETAILED DESCRIPTION OF THE INVENTION

“Isolated” means separated from its natural environment.

“Polynucleotide” generally relates to polyribonucleotides and polydeoxyribonucleotides, wherein the RNA or DNA may be unmodified or modified.

“Polypeptides” are taken to mean peptides or proteins, which contain two or more amino acids connected by peptide bonds.

The polypeptides according to the invention include the polypeptides according to SEQ ID no. 3 and SEQ ID no. 5 and according to SEQ ID no. 7, in particular those having the biological activity of succinate dehydrogenase and also those which are at least 70% identical to the polypeptide according to SEQ ID no. 3 and SEQ ID no. 5 and SEQ ID no. 7, preferably at least 80% and in particular 90% to 95% identical to the polypeptide according to SEQ ID no. 3 and SEQ ID no. 5 and SEQ ID no. 7 and exhibit the stated activity.

The invention furthermore relates to a process for the fermentative production of L-amino acids, in particular lysine, using coryneform bacteria, which in particular already produce the L-amino acids, in particular L-lysine, and in which the nucleotide sequences which code for the sdhC gene and/or the sdhA gene and/or the sdhB gene are attenuated, in particular are expressed at a low level.

In this connection, the term “attenuation” means reducing or suppressing the intracellular activity of one or more enzymes (proteins) in a microorganism, which enzymes are coded by the corresponding DNA, for example by using a weak promoter or a gene or allele which codes for a corresponding enzyme which has a low activity or inactivates the corresponding gene or enzyme (protein) and optionally by combining these measures.

The microorganisms, provided by the present invention, may produce L-amino acids, in particular L-lysine, from glucose, sucrose, lactose, fructose, maltose, molasses, starch, cellulose or from glycerol and ethanol. The microorganisms may comprise representatives of the coryneform bacteria in particular of the genus Corynebacterium. Within the genus Corynebacterium, the species Corynebacterium glutamicum may be particularly mentioned. It is known for its ability to produce L-amino acids.

Suitable strains of the genus Corynebacterium, in particular of the species Corynebacterium glutamicum, are especially the known wild type strains

Corynebacterium glutamicum ATCC13032

Corynebacterium acetoglutamicum ATCC15806

Corynebacterium acetoacidophilum ATCC13870

Corynebacterium melassecola ATCC17965

Corynebacterium thermoaminogenes FERM BP-1539

Brevibacterium flavum ATCC14067

Brevibacterium lactofermentum ATCC13869 and

Brevibacterium divaricatum ATCC14020

and L-amino acid producing mutants or strains produced therefrom,

such as for example the L-lysine producing strains

Corynebacterium glutamicum FERM-P 1709

Brevibacterium flavum FERM-P 1708

Brevibacterium lactofermentum FERM-P 1712

Corynebacterium glutamicum FERM-P 6463

Corynebacterium glutamicum FERM-P 6464 and

Corynebacterium glutamicum DSM 5714

The inventors succeeded in isolating the novel genes sdhC, sdhA and sdhB, which code for the enzyme succinate dehydrogenase (EC 1.3.99.1) I.B.R., from C. glutamicum.

The sdhC and/or sdhA gene and/or sdhB gene or also other genes are isolated from C. glutamicum by initially constructing a gene library of this microorganism in E. coli. The construction of gene libraries is described in generally known textbooks and manuals. Examples which may be mentioned are the textbook by Winnacker, Gene und Klone, Eine Einführung in die Gentechnologie (Verlag Chemie, Weinheim, Germany, 1990) I.B.R. or the manual by Sambrook et al., Molecular Cloning, A Laboratory Manual (Cold Spring Harbor Laboratory Press, 1989) I.B.R.

One very well known gene library is that of E. coli K-12 strain W3110, which was constructed by Kohara et al. (Cell 50, 495-508 (1987)) I.B.R. in λ-vectors. Bathe et al. (Molecular and General Genetics, 252:255-265, 1996) I.B.R. describe a gene library of C. glutamicum ATCC13032, which was constructed using the cosmid vector SuperCos I (Wahl et al., 1987, Proceedings of the National Academy of Sciences USA, 84:2160-2164) I.B.R. in E. coli K-12 strain NM554 (Raleigh et al., 1988, Nucleic Acids Research 16:1563-1575) I.B.R. Börmann et al. (Molecular Microbiology 6(3), 317-326, 1992)) I.B.R. also describe a gene library of C. glutamicum ATCC 13032, using cosmid pHC79 (Hohn and Collins, Gene 11, 291-298 (1980)) I.B.R. O'Donohue (The Cloning and Molecular Analysis of Four Common Aromatic Amino Acid Biosynthetic Genes from Corynebacterium glutamicum. Ph.D. Thesis, National University of Ireland, Galway, 1997) I.B.R. describes the cloning of C. glutamicum genes using the λ Zap Expression system described by Short et al. (Nucleic Acids Research, 16: 7583) I.B.R.

A gene library of C. glutamicum in E. coli may also be produced using plasmids such as pBR322 (Bolivar, Life Sciences, 25, 807-818 (1979)) I.B.R. or pUC9 (Vieira et al., 1982, Gene, 19:259-268) I.B.R. Suitable hosts are in particular those E. coli strains with restriction and recombination defects, such as for example strain DH5a (Jeffrey H. Miller: “A Short Course in Bacterial Genetics, A Laboratory Manual and Handbook for Escherichia coli and Related Bacterial”, Cold Spring Harbor Laboratory Press, 1992) I.B.R.

The long DNA fragments cloned with the assistance of cosmids or other λ vectors may then in turn be sub-cloned in conventional vectors suitable for DNA sequencing. DNA sequencing methods are described inter alia in Sanger et al. (Proceedings of the National Academy of Sciences of the United States of America USA, 74:5463-5467, 1977) I.B.R.

The resultant DNA sequences (or related protein sequences) may then be investigated using known algorithms or sequence analysis programs, for example Staden's program (Nucleic Acids Research 14, 217-232(1986)) I.B.R., Butler's GCG program (Methods of Biochemical Analysis 39, 74-97 (1998)) I.B.R., Pearson & Lipman's FASTA algorithm (Proceedings of the National Academy of Sciences U.S. Pat. No. 85,2444-2448 (1988)) I.B.R. or Altschul et al.'s BLAST algorithm (Nature Genetics 6, 119-129 (1994)) I.B.R. and compared with the sequence entries available in publicly accessible databases. Publicly accessible nucleotide sequence databases are, for example, the European Molecular Biology Laboratory database (EMBL, Heidelberg, Germany) I.B.R. (in the entirety as of Dec. 8, 2000) or the National Center for Biotechnology Information database (NCBI, Bethesda, Md., USA) I.B.R. (in the entirety as of Dec. 8, 2000).

The novel DNA sequence from C. glutamicum which codes for the sdhC gene and the sdhA gene and the sdhB gene and, as SEQ ID no. 1, is provided by the present invention, was obtained in this manner. The amino acid sequence of the corresponding proteins was furthermore deduced from the above DNA sequence using the methods described above. SEQ ID no. 3, SEQ ID no. 5 and SEQ ID no. 7 show the resultant amino acid sequences of the sdhC, sdhA and sdhB gene product.

Coding DNA sequences arising from SEQ ID no. 1 due to the degeneracy of the genetic code are also provided by the present invention. In a similar contect, conservative substitutions of amino acids in proteins, for example the substitution of glycine for alanine or of aspartic acid for glutamic acid, are known by the skilled artisan as “sense mutations”, which result in no fundamental change in activity of the protein. These types of changes are functionally neutral.

It is furthermore known that changes to the N and/or C terminus of a protein do not substantially impair or may even stabilise the function thereof. The person skilled in the art will find information in this connection inter alia in Ben-Bassat et al. (Journal of Bacteriology 169:751-757 (1987)) I.B.R., in O'Regan et al. (Gene 77:237-251 (1989)) I.B.R., in Sahin-Toth et al. (Protein Sciences 3:240-247 (1994)) I.B.R., in Hochuli et al. (Bio/Technology 6:1321-1325 (1988)) I.B.R. and in known textbooks of genetics and molecular biology. Amino acid sequences arising in a corresponding manner from SEQ ID no. 1 and DNA sequences, which code for these amino acid sequences, are also provided by the present invention.

DNA sequences which hybridize with SEQ ID no. 1 or parts of SEQ ID no. 1 are similarly provided by the invention. Finally, DNA sequences produced by the polymerase chain reaction (PCR) using primers obtained from SEQ ID no. 1 are also provided by the present invention.

The person skilled in the art can find instructions for identifying DNA sequences by means of hybridization, inter alia, in the manual “The DIG System Users Guide for Filter Hybridization” from Boehringer Mannheim GmbH (Mannheim, Germany, 1993) I.B.R. and in Liebl et al. (International Journal of Systematic Bacteriology (1991) 41: 255-260) I.B.R. The person skilled in the art may find instructions for amplifying DNA sequences using the polymerase chain reaction (PCR) inter alia in the manual by Gait, Oligonucleotide synthesis: a practical approach (IRL Press, Oxford, UK, 1984) I.B.R. and in Newton & Graham, PCR (Spektrum Akademischer Verlag, Heidelberg, Germany, 1994) I.B.R.

The inventors discovered that coryneform bacteria produce L-amino acids, in particular L-lysine, in an improved manner once the sdhC and/or sdhA and/or sdhB gene has been attenuated.

Attenuation may be achieved by reducing or suppressing either expression of the sdhC and/or sdhA and/or sdhB gene or the catalytic properties of the enzyme proteins. Both measures may optionally be combined.

Reduced gene expression may be achieved by appropriate control of the culture or by genetic modification (mutation) of the signal structures for gene expression. Signal structures for gene expression are, for example, repressor genes, activator genes, operators, promoters, attenuators, ribosome binding sites, the start codon and terminators. The person skilled in the art will find information in this connection for example in patent application WO 96/15246 I.B.R., in Boyd & Murphy (Journal of Bacteriology 170: 5949 (1988)) I.B.R., in Voskuil & Chambliss (Nucleic Acids Research 26: 3548 (1998)) I.B.R., in Jensen & Hammer (Biotechnology and Bioengineering 58: 191 (1998)) I.B.R., in Patek et al. (Microbiology 142: 1297 (1996)) I.B.R. and in known textbooks of genetics and molecular biology, such as for example the textbook by Knippers (“Molekulare Genetik”, 6th edition, Georg Thieme Verlag, Stuttgart, Germany, 1995) I.B.R. or by Winnacker (“Gene und Klone”, VCH Verlagsgesellschaft, Weinheim, Germany, 1990) I.B.R.

Mutations which give rise to a change or reduction in the catalytic properties of enzyme proteins are known from the prior art; examples which may be mentioned are the papers by Qiu and Goodman (Journal of Biological Chemistry 272: 8611-8617 (1997)) I.B.R., Sugimoto et al. (Bioscience Biotechnology and Biochemistry 61: 1760-1762 (1997)) I.B.R. and Möckel (“Die Threonindehydratase aus Corynebacterium glutamicum: Aufhebung der allosterischen Regulation und Struktur des Enzyms”, Berichte des Forschungszentrums Jülichs, Jül-2906, ISSN09442952, Jülich, Germany, 1994) I.B.R. Summary presentations may be found in known textbooks of genetics and molecular biology such as, for example, the textbook by Hagemann (“Allgemeine Genetik”, Gustav Fischer Verlag, Stuttgart, 1986) I.B.R.

Mutations, which may be considered, are transitions, transversions, insertions and deletions. Depending upon the effect of exchanging the amino acids upon enzyme activity, the mutations are known as missense mutations or nonsense mutations. Insertions or deletions of at least one base pair in a gene give rise to frame shift mutations, as a result of which the incorrect amino acids are inserted or translation terminates prematurely. Deletions of two or more codons typically result in a complete breakdown of enzyme activity. Instructions for producing such mutations belong to the prior art and may be found in known textbooks of genetics and molecular biology, such as for example the textbook by Knippers (“Molekulare Genetik”, 6th edition, Georg Thieme Verlag, Stuttgart, Germany, 1995) I.B.R., by Winnacker (“Gene und Klone”, VCH Verlagsgesellschaft, Weinheim, Germany, 1990) I.B.R. or by Hagemann (“Allgemeine Genetik”, Gustav Fischer Verlag, Stuttgart, 1986) I.B.R.

One common method of mutating genes of C. glutamicum is the method of gene disruption and gene replacement described by Schwarzer & Pühler (Bio/Technology 9, 84-87 (1991)) I.B.R. In the gene disruption method, a central portion of the coding region of the gene under consideration is cloned into a plasmid vector which may replicate in a host (typically E. coli), but not in C. glutamicum. Vectors which may be considered are, for example, pSUP301 (Simon et al., Bio/Technology 1, 784-791 (1983)) I.B.R., pK18mob or pK19mob (Schafer et al., Gene 145, 69-73 (1994)) I.B.R., pK18mobsacB or pK19mobsacB (Jäger et al., Journal of Bacteriology 174: 5462-65 (1992)) I.B.R., PGEM-T (Promega Corporation, Madison, Wis., USA), pCR2.1-TOPO (Shuman (1994)) I.B.R. Journal of Biological Chemistry 269:32678-84 I.B.R.; U.S. Pat. No. 5,487,993) I.B.R., pCR®Blunt (Invitrogen, Groningen, Netherlands; Bernard et al., Journal of Molecular Biology, 234: 534-541 (1993)) I.B.R. or pEM1 (Schrumpf et al., 1991, Journal of Bacteriology 173:4510-4516) I.B.R.

The plasmid vector that contains the central portion of the coding region of the gene is then transferred into the desired strain of C. glutamicum by conjugation or transformation. The conjugation method is described, for example, in Schäfer et al. (Applied and Environmental Microbiology 60, 756-759 (1994)) I.B.R. Transformation methods are described, for example, in Thierbach et al. (Applied Microbiology and Biotechnology 29, 356-362 (1988)) I.B.R., Dunican and Shivnan (Bio/Technology 7, 1067-1070 (1989)) I.B.R. and Tauch et al. (FEMS Microbiological Letters 123, 343-347 (1994)) I.B.R.

After homologous recombination by means of “crossing over”, the coding region of the gene concerned is interrupted by the vector sequence and two incomplete alleles are obtained each of which lacks the 3′ or 5′ end. This method has been described, for example by Fitzpatrick et al. (Applied Microbiology and Biotechnology 42, 575-580 (1994)) I.B.R. for suppressing the recA gene of C. glutamicum. The sdhC and/or sdhA and/or sdhB genes may be suppressed in this manner

In the gene replacement method, a mutation, such as for example a deletion, insertion or base replacement, is produced in vitro in the gene under consideration. The resultant allele is in turn cloned into a vector, which is non-replicative in C. glutamicum, which vector is then transferred into the desired host of C. glutamicum by In transformation or conjugation. After homologous recombination by means of a first “crossing over”, which effects integration, and a suitable second “crossing over”, which effects excision, in the target gene or target sequence, the mutation or allele is incorporated. This method has been used, for example by Peters-Wendisch (Microbiology 144, 915-927 (1998)) I.B.R. to suppress the pyc gene of C. glutamicum by a deletion. A deletion, insertion or base replacement may be incorporated into the sdhC and/or sdhA and/or sdhB gene in this manner.

The present invention accordingly also provides a process for the fermentative production of L-amino acids, in particular L-lysine, in which either a strain transformed with a plasmid vector is used and the plasmid vector bears nucleotide sequences for the genes coding for the enzyme succinate dehydrogenase or the strain bears a deletion, insertion or base replacement in the sdhC and/or sdhA and/or sdhB gene.

Processes for the fermentative production of L-amino acids, in particular L-lysine, contain the following steps:

a) fermentation of the L-amino acid producing coryneform bacteria in which at least one of the genes, selected from among the genes coding for the enzyme succinate dehydrogenase and the subunits A, B and C thereof, is attenuated,

b) accumulation of the L-amino acid in the medium or in the cells of the bacteria and

c) isolation of the L-amino acid.

It may additionally be advantageous for the production of L-amino acids, in particular L-lysine, in addition to attenuating the sdhC and/or sdhA and/or sdhB gene, to amplify, in particular to overexpress, one or more enzymes of the particular biosynthetic pathway, of glycolysis, of anaplerotic metabolism, of the citric acid cycle or of amino acid export.

Thus, for example, for the production of L-lysine

the dapA gene (EP-B 0 197 335) I.B.R., which codes for dihydropicolinate synthase, may simultaneously be overexpressed, or

the gap gene, which codes for glyceraldehyde 3-phosphate dehydrogenase (Eikmanns (1992), Journal of Bacteriology 174:6076-6086) I.B.R., may simultaneously be overexpressed or

the pyc gene (DE-A-198 31 609) I.B.R. which codes for pyruvate carboxylase may simultaneously be overexpressed or

the mqo gene (Molenaar et al., European Journal of Biochemistry 254, 395-403 (1998)) I.B.R., which codes for malate:quinone oxidoreductase, may simultaneously be overexpressed, or

the lysE gene (DE-A-195 48 222) I.B.R., which codes for lysine export, may simultaneously be overexpressed.

It may furthermore be advantageous for the production of amino acids, in particular L-lysine, simultaneously to attenuate

the pck gene which codes for phosphoenolpyruvate carboxykinase (DE 199 50 409.1, DSM 13047) I.B.R. and/or

the pgi gene which codes for glucose 6-phosphate isomerase (U.S. Ser. No. 09/396,478, DSM 12969) I.B.R.

It may furthermore be advantageous for the production of L-amino acids, in particular L-lysine, in addition to attenuating the sdhC and/or sdhA and/or sdhB gene, to suppress unwanted secondary reactions (Nakayama: “Breeding of Amino Acid Producing Micro-organisms”, in: Overproduction of Microbial Products, Krumphanzl, Sikyta, Vanek (eds.), Academic Press, London, UK, 1982) I.B.R.

The microorganisms containing the polynucleotide according to claim 1 are also provided by the invention and may be cultured continuously or discontinuously using the batch process or the fed batch process or repeated fed batch process for the purpose of producing L-amino acids, in particular L-lysine. A summary of known culture methods is given in the textbook by Chmiel (Bioprozesstechnik 1. Einführung in die Bioverfahrenstechnik (Gustav Fischer Verlag, Stuttgart, 1991)) I.B.R. or in the textbook by Storhas (Bioreaktoren und periphere Einrichtungen (Vieweg Verlag, Braunschweig/Wiesbaden, 1994)) I.B.R.

The culture medium to be used must adequately satisfy the requirements of the particular strains. Culture media for various microorganisms are described in “Manual of Methods for General Bacteriology” from the American Society for Bacteriology (Washington D.C., USA, 1981) I.B.R. Carbon sources which may be used include sugars and carbohydrates, such as for example glucose, sucrose, lactose, fructose, maltose, molasses, starch and cellulose, oils and fats, such as for example soya oil, sunflower oil, peanut oil and coconut oil, fatty acids, such as for example palmitic acid, stearic acid and linoleic acid, alcohols, such as for example glycerol and ethanol, and organic acids, such as for example acetic acid. These substances may be used individually or as a mixture.

Nitrogen sources which may be used comprise organic compounds containing nitrogen, such as peptones, yeast extract, meat extract, malt extract, corn steep liquor, soya flour and urea or inorganic compounds, such as ammonium sulfate, ammonium chloride, ammonium phosphate, ammonium carbonate and ammonium nitrate. The nitrogen sources may be used individually or as a mixture.

Phosphorus sources, which may be used, are phosphoric acid, potassium dihydrogen phosphate or dipotassium hydrogen phosphate or the corresponding salts containing sodium. The culture medium has additionally to contain salts of metals, such as magnesium sulfate or iron sulfate for example, which are necessary for growth.

Finally, essential growth-promoting substances such as amino acids and vitamins may also be used in addition to the above-stated substances. Suitable precursors may furthermore be added to the culture medium. The stated feed substances may be added to the culture as a single batch or be fed appropriately during culturing.

Basic compounds, such as sodium hydroxide, potassium hydroxide, ammonia or ammonia water, or acidic compounds, such as phosphoric acid or sulfuric acid, are used appropriately to control the pH of the culture. Foaming may be controlled by using antifoaming agents such as fatty acid polyglycol esters for example.

Plasmid stability may be maintained by the addition to the medium of suitable selectively acting substances, for example antibiotics. Oxygen or gas mixtures containing oxygen, such as for example air, are introduced into the culture in order to maintain aerobic conditions. The temperature of the culture is normally from 20° C. to 45° C. and preferably from 25° C. to 40° C. The culture is continued until a maximum quantity of the desired product has been formed. This aim is normally achieved within 10 to 160 hours.

Methods for determining L-amino acids are known from the prior art. Analysis may proceed by anion exchange chromatography with subsequent ninhydrin derivatisation, as described in Spackman et al. (Analytical Chemistry, 30, (1958), 1190) I.B.R. or by reversed phase HPLC, as described in Lindroth et al. (Analytical Chemistry (1979) 51: 1167-1174) I.B.R.

### EXAMPLES

The present invention is illustrated in greater detail by the following practical examples.

### Example 1

Production of a Genomic Cosmid Gene Library From Corynebacterium glutamicum ATCC13032

Chromosomal DNA from Corynebacterium glutamicum ATCC 13032 was isolated as described in Tauch et al., (1995, Plasmid 33:168-179) I.B.R. and partially cleaved with the restriction enzyme Sau3AI (Amersham Pharmacia, Freiburg, Germany, product description Sau3AI, code no. 27-0913-02). The DNA fragments were dephosphorylated with shrimp alkaline phosphatase (Roche Molecular Biochemicals, Mannheim, Germany, product description SAP, code no. 1758250).

The DNA of cosmid vector SuperCos1 (Wahl et al. (1987) Proceedings of the National Academy of Sciences USA 84:2160-2164) I.B.R., purchased from Stratagene (La Jolla, USA, product description SuperCos1 Cosmid Vector Kit, code no. 251301) was cleaved with the restriction enzyme XbaI (Amersham Pharmacia, Freiburg, Germany, product description XbaI, code no. 27-0948-02) and also dephosphorylated with shrimp alkaline phosphatase.

The cosmid DNA was then cleaved with the restriction enzyme BamHI (Amersham Pharmacia, Freiburg, Germany, product description BamHI, code no. 27-0868-04). Cosmid DNA treated in this manner was mixed with the treated ATCC13032 DNA and the batch was treated with T4 DNA ligase (Amersham Pharmacia, Freiburg, Germany, product description T4 DNA Ligase, code no. 27-0870-04). The ligation mixture was then packed in phages using Gigapack II XL Packing Extracts (Stratagene, La Jolla, USA, product description Gigapack II XL Packing Extract, code no. 200217).

E. coli strain NM554 (Raleigh et al. 1988, Nucleic Acid Res. 16:1563-1575) I.B.R. was infected by suspending the cells in 10 mM MgSO₄ and mixing them with an aliquot of the phage suspension. The cosmid library was infected and titred as described in Sambrook et al. (1989, Molecular Cloning: A laboratory Manual, Cold Spring Harbor) I.B.R., the cells being plated out on LB agar (Lennox, 1955, Virology, 1:190) I.B.R. with 100 μg/ml of ampicillin. After overnight incubation at 37° C., individual recombinant clones were selected.

### Example 2

Isolation and Sequencing of the sdhC, sdhA and sdhB Genes

Cosmid DNA from an individual colony was isolated in accordance with the manufacturer's instructions using the Qiaprep Spin Miniprep Kit (product no. 27106, Qiagen, Hilden, Germany) and partially cleaved with the restriction enzyme Sau3AI (Amersham Pharmacia, Freiburg, Germany, product description Sau3AI, product no. 27-0913-O₂). The DNA fragments were dephosphorylated with shrimp alkaline phosphatase (Roche Molecular Biochemicals, Mannheim, Germany, product description SAP, product no. 1758250).

Once separated by gel electrophoresis, the cosmid fragments of a size of 1500 to 2000 bp were isolated using the QiaExII Gel Extraction Kit (product no. 20021, Qiagen, Hilden, Germany). The DNA of the sequencing vector pZero-1 purchased from Invitrogen (Groningen, Netherlands, product description Zero Background Cloning Kit, product no. K2500-01) was cleaved with the restriction enzyme BamHI (Amersham Pharmacia, Freiburg, Germany, product description BamHI, product no. 27-0868-04).

Ligation of the cosmid fragments into the sequencing vector pZero-1 was performed as described by Sambrook et al. (1989, Molecular Cloning: A laboratory Manual, Cold Spring Harbor) I.B.R., the DNA mixture being incubated overnight with T4 ligase (Pharmacia Biotech, Freiburg, Germany). This ligation mixture was then electroporated into the E. coli strain DH5αMCR (Grant, 1990, Proceedings of the National Academy of Sciences U.S.A., 87:4645-4649) I.B.R. (Tauch et al. 1994, FEMS Microbiol Letters, 123:343-7) I.B.R. and plated out onto LB agar (Lennox, 1955, Virology, 1:190) I.B.R. with 50 μg/ml of Zeocin. Plasmids of the recombinant clones were prepared using the Biorobot 9600 (product no. 900200, Qiagen, Hilden, Germany).

Sequencing was performed using the dideoxy chain termination method according to Sanger et al. (1977, Proceedings of the National Academies of Sciences U.S.A., 74:5463-5467) I.B.R. as modified by Zimmermann et al. (1990, Nucleic Acids Research, 18:1067) I.B.R. The “RR dRhodamin Terminator Cycle Sequencing Kit” from PE Applied Biosystems (product no. 403044, Weiterstadt, Germany) was used.

Separation by gel electrophoresis and analysis of the sequencing reaction was performed in a “Rotiphorese NF” acrylamide/bisacrylamide gel (29:1) (product no. A124.1, Roth, Karlsruhe, Germany) using the “ABI Prism 377” -sequencer from PE Applied Biosystems (Weiterstadt, Germany).

The resultant raw sequence data were then processed using the Staden software package (1986, Nucleic Acids Research, 14:217-231) I.B.R., version 97-0. The individual sequences of the pzero 1 derivatives were assembled into a cohesive contig. Computer-aided coding range analysis was performed using XNIP software (Staden, 1986, Nucleic Acids Research, 14:217-231) I.B.R. Further analysis was performed using the “BLAST search programs” (Altschul et al., 1997, Nucleic Acids Research, 25:3389-3402) I.B.R., against the non-redundant database of the “National Center for Biotechnology Information” (NCBI, Bethesda, Md., USA) I.B.R.

The resultant nucleotide sequence is stated in SEQ ID no. 1. Analysis of the nucleotide sequence revealed an open reading frame of 879 base pairs, which was designated the sdhC gene and an open reading frame of 1875 base pairs, which was designated sdhA and an open reading frame of 852 base pairs, which was designated the sdhB gene. The sdhC gene codes for a polypeptide of 293 amino acids, which is shown in SEQ ID no. 3. The sdhA gene codes for a polypeptide of 625 amino acids, which is shown in SEQ ID no. 5. The sdhB gene codes for a polypeptide of 284 amino acids, which is shown in SEQ ID no. 7.

### Example 3

Production of an Integration Vector Integration Mutagenesis of the sdhA Gene

Chromosomal DNA was isolated from strain ATCC 13032 using the method of Eikmanns et al. (Microbiology 140: 1817-1828 (1994)) I.B.R. On the basis of the sequence of the sdhA gene for C. glutamicum known from Example 2, the following oligonucleotides were selected for the polymerase chain reaction:

sdhA-in1:

5′CGT CAT TGT CAC CGA ACG TA 3′

sdhA-in2:

5′TCG TTG AAG TCA GTC CAG AG 3′

The stated primers were synthesised by the company MWG Biotech (Ebersberg, Germany) and the PCR reaction performed in accordance with the standard PCR method of Innis et al. (PCR protocols. A guide to methods and applications, 1990, Academic Press) I.B.R. using Pwo polymerase from Boehringer Mannheim (Germany, production description Pwo DNA Polymerase, product no. 1 644 947). By means of the polymerase chain reaction, the primers permit the amplification of an approx. 0.67 kb internal fragment of the sdhA gene. The product amplified in this manner was verified electrophoretically in a 0.8% agarose gel.

The amplified DNA fragment was ligated into the vector pCRBlunt® II (Bernard et al., Journal of Molecular Biology, 234:534-541) I.B.R. using the Zero Blunt™ Kit from Invitrogen Corporation (Carlsbad, Calif., USA; catalogue number K2700-20).

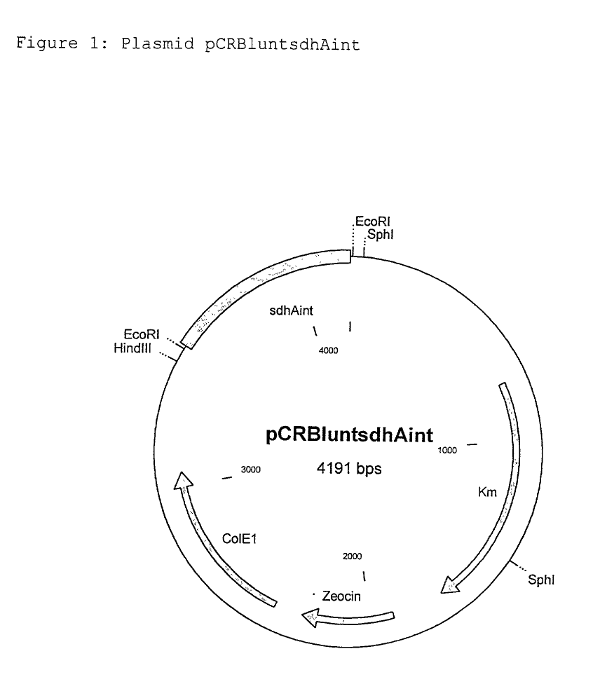

The E. coli strain TOP10 was then electroporated with the ligation batch (Hanahan, in DNA cloning. A practical approach. Vol.I. IRL-Press, Oxford, Washington D.C., USA, 1985) I.B.R. Plasmid-bearing cells were selected by plating the transformation batch out onto LB agar (Sambrook et al., Molecular cloning: a laboratory manual. 2^{nd} Ed. Cold Spring Harbor Laboratory Press, Cold Spring Harbor, N.Y., 1989) I.B.R. which had been supplemented with 25 mg/l of kanamycin. Plasmid DNA was isolated from a transformant using the QIAprep Spin Miniprep Kit from Qiagen and verified by restriction with the restriction enzyme EcoRI and subsequent agarose gel electrophoresis (0.8%). The plasmid was named pCRBluntsdhAint and is shown in FIG. 1.

### Example 4

Integration Mutagenesis of the sdhA Gene into the Strain DSM 5715

The vector named pCRBluntsdhAint in Example 3 was electroporated into Corynebacterium glutamicum DSM 5715 using the electroporation method of Tauch et al. (FEMS Microbiological Letters, 123:343-347 (1994)) I.B.R. Strain DSM 5715 is described in EP-B-0435132 I.B.R. The vector pCRBluntsdhAint cannot independently replicate in DSM 5715 and is only retained in the cell if it has been integrated into the chromosome of DSM 5715. Clones with pCRBluntsdhAint integrated into the chromosome were selected by plating the electroporation batch out onto LB agar (Sambrook et al., Molecular cloning: a laboratory manual. 2,1^{nd} Ed. Cold Spring Harbor Laboratory Press, Cold Spring Harbor, N.Y.) I.B.R. which had been supplemented with 15 mg/l of kanamycin.

Integration was detected by labelling the sdhAint fragment with the Dig hybridisation kit from Boehringer using the method according to “The DIG System Users Guide for Filter Hybridisation” from Boehringer Mannheim GmbH (Mannheim, Germany, 1993) I.B.R.

Chromosomal DNA of a potential integrant was isolated using the method according to Eikmanns et al. (Microbiology 140: 1817-1828 (1994)) I.B.R. and cut in each case with the restriction enzymes SphI and HindIII. The resultant fragments were separated by means of agarose gel electrophoresis and hybridised at 68° C. using the Dig hybridisation kit from Boehringer. The plasmid named pCRBluntsdhAint in Example 3 had been inserted within the chromosomal sdhA gene in the chromosome of DSM5715. The strain was designated DSM5715::pCRBluntsdhAint.

### Example 5

Production of L-Glutamic Acid with Strain DSM5715::pCRBluntsdhAint

The C. glutamicum strain DSM5715::pCRBluntsdhAint obtained in Example 4 was cultured in a nutrient medium suitable for the production of glutamic acid and the glutamic acid content of the culture supernatant was determined.

To this end, the strain was initially incubated for 24 hours at 33° C. on an agar plate with the appropriate antibiotic (brain/heart agar with kanamycin (25 mg/l)). Starting from this agar plate culture, a preculture was inoculated (10 ml of medium in a 100 ml Erlenmeyer flask). The complete medium CgIII was used as the medium for this preculture.

### Medium Cg III

Medium Cg III

| | | |
|---|---|---|
| NaCl | 2.5 | g/l |
| Bacto peptone | 10 | g/l |
| Bacto yeast extract | 10 | g/l |
| Glucose (separately autoclaved) | 2% | (w/v) |

The pH value was adjusted to pH 7.4.

Kanamycin (25 mg/l) was added to this medium. The preculture was incubated for 16 hours at 33° C. on a shaker at 240 rpm. A main culture was inoculated from this preculture, such that the initial OD (660 nm) of the main culture was 0.1 OD. Medium MM was used for the main culture.

Medium MM

| | | |
|---|---|---|
| CSL (Corn Steep Liquor) | 5 | g/l |
| MOPS (morpholinopropanesulfonic | 20 | g/l |
| acid) | | |
| Sodium acetate (sterile-filtered) | 20 | g/l |
| Salts: | | |
| (NH₄)₂SO₄) | 25 | g/l |
| KH₂PO₄ | 0.1 | g/l |
| MgSO₄ * 7 H₂O | 1.0 | g/l |
| CaCl₂ * 2 H₂O | 10 | mg/l |
| FeSO₄ * 7 H₂O | 10 | mg/l |
| MnSO₄ * H₂O | 5.0 | mg/l |
| Biotin (sterile-filtered) | 0.3 | mg/l |
| Thiamine * HCl (sterile-filtered) | 0.2 | mg/l |
| Leucine (sterile-filtered) | 0.1 | g/l |
| CaCO₃ | 25 | g/l |

CSL, MOPS and the salt solution are adjusted to pH 7 with ammonia solution and autoclaved. The sterile substrate and vitamin solutions, together with the dry-autoclaved CaCO₃ are then added.

Culturing is performed in a volume of 10 ml in a 100 ml Erlenmeyer flask with flow spoilers. Kanamycin (25 mg/l) was added. Culturing was performed at 330C and 80% atmospheric humidity.

After 24 hours, the OD was determined at a measurement wavelength of 660 nm using a Biomek 1000 (Beckmann Instruments GmbH, Munich). The quantity of glutamic acid formed was determined using an amino acid analyser from Eppendorf-BioTronik (Hamburg, Germany) by ion exchange chromatography and post-column derivatisation with ninhydrin detection.

Table 1 shows the result of the test.

TABLE 1

| | | L-glutamic |
|---|---|---|
| Strain | OD(660) | acid (mg/l) |
| DSM5715 | 6.6 | 41 |
| DSM5715: :pCRBluntsdhAint | 5.1 | 155 |

The abbreviations and names are defined as follows. The stated base pair figures are approximate values obtained within the framework of measurement reproducibility.

| | |
|---|---|
| Km: | Kanamycin resistance gene |
| Zeocin: | Zeocin resistance gene |
| HindIII: | Restriction site of the restriction enzyme |
| HindIII | |
| SphI: | Restriction site of the restriction enzyme SphI |
| EcoRI: | Restriction site of the restriction enzyme EcoRI |
| sdhAint: | internal fragment of the sdhA gene |
| ColE1 ori: | Replication origin of the plasmid ColE1 |

Further variations and modifications of the present invention will be apparent to those skilled in the art from a reading of the foregoing and are encompassed by the claims appended hereto.

German patent application 199 59 650.6 I.B.R. is relied upon and incorporated herein by reference.

We claim:

1. An isolated polynucleotide from coryneform bacteria containing a polynucleotide sequence selected from the group consisting of:
a) a polynucleotide which is at least 70% identical to a polynucleotide which codes for a polypeptide containing the amino acid sequence of SEQ ID no. 3,
b) a polynucleotide which is at least 70% identical to a polynucleotide which codes for a polypeptide containing the amino acid sequence of SEQ ID no. 5,
c) a polynucleotide which is at least 70% identical to a polynucleotide which codes for a polypeptide containing the amino acid sequence of SEQ ID no. 7,
d) a polynucleotide which codes for a polypeptide which contains an amino acid sequence which is at least 70% identical to the amino acid sequence of SEQ ID no. 3,
e) a polynucleotide which codes for a polypeptide which contains an amino acid sequence which is at least 70% identical to the amino acid sequence of SEQ ID no. 5,
f) a polynucleotide which codes for a polypeptide which contains an amino acid sequence which is at least 70% identical to the amino acid sequence of SEQ ID no. 7,
g) a polynucleotide which is complementary to the polynucleotides of a), b), c), d), e) or f) and,
h) a polynucleotide containing at least 15 successive nucleotides of the polynucleotide sequence of a), b), c), d), e) or f).

2. The polynucleotide according to claim 1,
wherein the polynucleotide is a recombinant DNA replicable in coryneform bacteria.

3. A polynucleotide according to claim 1,
wherein the polynucleotide is an RNA.

4. The replicable DNA according to claim 2, containing
(i) the nucleotide sequence shown in SEQ ID no. 1, or
(ii) at least one sequence which matches the sequence (i) within the degeneration range of the genetic code, or
(iii) at least one sequence which hybridizes with the complementary sequence to sequence (i) or (ii) and optionally
(iv) functionally neutral sense mutations in (i).

5. The polynucleotide sequence according to claim 2 which codes for a polypeptide which contains the amino acid sequence as shown in SEQ ID no. 2.

6. A vector containing the polynucleotide sequence according to claim 1.

7. A vector containing a polynucleotide sequence, wherein the polynucleotide contains at least 15 successive nucleotides of the polynucleotide sequence of claim 1.

8. A coryneform bacterium containing a vector according to claim 6.

9. A process for the preparation of L-amino acids comprising:
fermenting an L-amino acid-producing bacteria in which at least one of the genes, selected from among the genes coding for the enzyme succinate dehydrogenase or one of the subunits A, B and C thereof, is attenuated.

10. The process according to claim 9, and further comprising accumulating the L-amino acid produced in the medium or in the cells of the bacteria.

11. The process according to claim 10, and further comprising isolating the L-amino acid.

12. The process according to claim 9, wherein bacteria is used in which a further gene of the biosynthetic pathway of the desired L-amino acid is enhanced.

13. The process according to claim 9, wherein bacteria is used in which a metabolic pathway which reduces formation of the L-amino acid is at least partially suppressed.

14. The process according to claim 9, wherein a bacteria strain, which is transformed with a plasmid vector, is utilized and the plasmid vector bears a nucleotide sequence of the gene coding for the enzyme succinate dehydrogenase or a functional equivalent thereof.

15. The process according to claim 9, wherein a coryneform bacteria is used which produces L-lysine.

16. The process according to claim 12, wherein a dapA gene or functional equivalent thereof, which codes for dihydropicolinate synthase or functional equivalent thereof, is simultaneously overexpressed.

17. The process according to claim 12, wherein a gap gene or functional equivalent thereof, which codes for glyceraldehyde 3-phosphate dehydrogenase or functional equivalent thereof, is simultaneously overexpressed.

18. The process according to claim 12, wherein a pyc gene or functional equivalent thereof, which codes for pyruvate carboxylase or functional equivalent thereof, is simultaneously overexpressed.

19. The process according to claim 12, wherein an mqo gene or functional equivalent thereof, which codes for malate quinone oxidoreductase or functional equivalent thereof, is simultaneously overexpressed.

20. The process according to claim 12, wherein a lysE gene or functional equivalent thereof, which codes for lysine export or functional equivalent thereof, is simultaneously overexpressed.

21. The process according to claim 13, wherein a pgi gene or functional equivalent thereof, which codes for glucose-6-phosphate isomerase or functional equivalent thereof, is simultaneously attenuated.

22. The process according to claim 13, wherein a pck gene or functional equivalent thereof, which codes for phosphoenolpyruvate carboxykinase or functional equivalent thereof, is simultaneously attenuated.
